(19) United States
(12) Patent Application Publication
(10) Pub. No.: US 2024/0395417 A1
(43) Pub. Date: Nov. 28, 2024

(54) SYSTEMS AND METHODS FOR DETERMINING READMISSION RATES

(71) Applicant: Optum, Inc., Minnetonka, MN (US)

(72) Inventors: Elan GADA, Dallas, TX (US); Richard YOUNG, Las Vegas, NV (US)

(21) Appl. No.: 18/323,518

(22) Filed: May 25, 2023

(51) Int. Cl. G16H 50/70; G06N 5/046; G16H 10/60

(52) U.S. Cl. CPC G16H 50/70; G06N 5/046; G16H 10/60; G16H 70/20

(57) ABSTRACT

Systems and methods include obtaining hospital admission data associated with a plurality of individuals, the hospital admission data including at least one indicator of a disease of interest and at least one admission date, determining a primary admission value based on the hospital admission data, determining a readmission value based on the hospital admission data, determining a disease-specific readmission rate based on the primary admission value and the readmission value, wherein the primary admission value and the readmission value are based on a common indicator of a disease of interest, and causing to output data associated with the disease-specific readmission rate via a graphical user interface of a user device.

### TECHNICAL FIELD

The present disclosure relates generally to data collection, data processing, and data analysis, and more particularly, to systems and methods for determining hospital readmission rates and trend predictions.

### BACKGROUND

Hospital readmissions are a significant contributor to rising healthcare costs. For example, a readmission following a premature discharge may incur greater costs than if a patient remains in the hospital for a longer amount of time. Yet, patients tend to be discharged as soon as possible for various reasons, e.g., costs, preventing opportunistic infections, etc. While some readmissions may be unavoidable for various reasons, many readmissions may be avoided and/or prevented under certain circumstances. Conventional methods for attempting to reduce hospital readmissions involve limited data, e.g., not real-time data, and often fail to take into account the various causes of readmissions that are often marked drivers of poor health outcomes and increased health costs.

Unless otherwise indicated herein, the materials described in this section are not prior art to the claims in this application and are not admitted to be prior art, or suggestions of the prior art, by inclusion in this section.

### SUMMARY

The present disclosure solves this problem and/or other problems described above or elsewhere in the present disclosure and improves the state of conventional healthcare applications.

In some embodiments, a computer-implemented method is disclosed. The method may include obtaining, by one or more processors, hospital admission data associated with a plurality of individuals, the hospital admission data including at least one indicator of a disease of interest and at least one admission date; determining, by the one or more processors, a primary admission value based on the hospital admission data; determining, by the one or more processors, a readmission value based on the hospital admission data; determining, by the one or more processors, a disease-specific readmission rate based on the primary admission value and the readmission value, wherein the primary admission value and the readmission value are based on a common indicator of a disease of interest; and causing to output, by the one or more processors, data associated with the disease-specific readmission rate via a graphical user interface of a user device.

In some embodiments, a system is disclosed. The system may include one or more storage devices each configured to store instructions; and one or more processors configured to execute the instructions to perform operations. The operations may include obtaining hospital admission data associated with a plurality of individuals, the hospital admission data including at least one indicator of a disease of interest and at least one admission date; determining a primary admission value based on the hospital admission data; determining a readmission value based on the hospital admission data; determining a disease-specific readmission rate based on the primary admission value and the readmission value, wherein the primary admission value and the readmission value are based on a common indicator of a disease of interest; and causing to output data associated with the disease-specific readmission rate via a graphical user interface of a user device.

In some embodiments, a non-transitory computer-readable medium comprising instructions that, when executed by one or more processors, cause the one or more processors to perform operations. The operations may include obtaining hospital admission data associated with a plurality of individuals, the hospital admission data including at least one indicator of a disease of interest and at least one admission date; determining a primary admission value based on the hospital admission data; determining a readmission value based on the hospital admission data; determining a disease-specific readmission rate based on the primary admission value and the readmission value, wherein the primary admission value and the readmission value are based on a common indicator of a disease of interest; and causing to output data associated with the disease-specific readmission rate via a graphical user interface of a user device.

It is to be understood that both the foregoing general description and the following detailed description are example and explanatory only and are not restrictive of the detailed embodiments, as claimed.

### BRIEF DESCRIPTION OF THE DRAWINGS

The accompanying drawings, which are incorporated in and constitute a part of this specification, illustrate various example embodiments and together with the description, serve to explain the principles of the disclosed embodiments.

FIG. 1 depicts an example environment for determining hospital readmission rates, according to some embodiments of the disclosure.

FIG. 2 depicts an example method for determining hospital readmission rates, according to some embodiments of the disclosure.

FIGS. 3A and 3B depict example methods for determining disease-specific and disease-any-cause hospital readmission rates, according to some embodiments of the disclosure.

FIG. 4 depicts an example machine learning training flow chart, according to some embodiments of the disclosure.

FIG. 5 illustrates an implementation of a computer system that executes techniques presented herein, according to some embodiments of the disclosure.

### DETAILED DESCRIPTION

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

Various non-limiting embodiments of the present disclosure will now be described to provide an overall understanding of the principles of the structure, function, and use of systems and methods disclosed herein for determining hospital readmission rates.

Hospital readmissions are a significant driver of raising healthcare costs. For example, readmissions are costly to patients, e.g., because of costs associated with multiple rounds of testing, increasing number of prescriptions, increasing prescription costs, etc., and also to hospitals, which may be fined under various government programs, e.g., the Centers for Medicare and Medicaid Services (CMS) Readmission Reduction Program, for having excess readmission rates.

Conventional methods for determining hospital admission rates have failed to accurately capture readmission values and/or rates, and to predict readmission, a key factor in preventing readmission. Under some conventional techniques, readmissions due to a second, a third, and any subsequent disease are excluded from consideration. For example, if readmissions for pulmonary emboli are tracked, readmissions for anxiety disorders are excluded. Additionally, under some conventional techniques, patients discharged “against medical advice” are excluded from readmission rate calculations, despite the fact that hospitals are penalized for excessive readmissions no matter the cause. In other words, the conventional technique of excluding patients discharged “against medical advice” produces a misleading value that hospitals and patients cannot rely upon for accuracy. Finally, under some conventional techniques, only readmissions that fall within a single calendar year, e.g., between January 1 and December 1 of a measurement year are considered in the calculation. This technique fails to capture the overall picture of a patient's readmission experience. Time-limiting the value to an arbitrary time excludes many readmission dates, which often fall outside of this range, but are pertinent to the calculation.

The techniques disclosed in the present disclosure aim to help minimize the problems discussed above, including reducing the cost of healthcare based on hospital readmissions. To accomplish this, hospital readmissions are determined in both disease-specific and disease-agnostic calculations, taking into account all relevant data including but not limited to disease diagnosis, prescriptions, admissions, readmissions, various time frames (e.g., 20 days, 30 days, 45 days, 60 days, etc.), etc. The generation of the datasets described herein advantageously improves data processing, as the typical size of initial datasets, e.g., raw disease-specific dataset, raw disease-any-cause dataset, etc., can be enormous. Removal of extraneous data in those datasets dramatically improves the speed and accuracy of subsequent analysis (e.g., analysis of the reduced dataset to determine readmission rates), as the computing power and resources required to perform the data analysis would be significantly reduced. Additionally or alternatively, at least one trend prediction is determined based on the data and/or the readmission rates. At least one trend prediction includes any of readmission prediction, disease tracking, cost prediction, etc. Advantageously, at least one trend prediction may be determined using at least one trained machine learning model, which advantageously account for myriad data that are relevant in determining the at least one trend prediction (e.g., a patient's admitting diagnosis, the readmission rate for a given diagnosis, etc.). Further advantageously, the machine learning techniques described herein improve feature extraction, data accuracy and reliability, patient predictions (e.g., readmission, outcome, etc.), etc.

Additionally, current data collection methods used for the readmission analysis could become more error-prone without direct communication between the data source (e.g., a hospital) and the system that performs the readmission analysis, as there could be multiple middlemen, billing claims generators, the insurance company, the payment processor, and other entities who interact with the data. Inaccurate data may lead to downstream consequences for the development of a machine learning algorithm. For example, the machine learning algorithm would make inaccurate predictions after relying on inaccurate data. This in turn could have clinical and financial consequences. For example, healthcare providers may end up spending time and resources on patients who are unlikely to readmit, while not focusing enough time and skill on those with a high likelihood of readmission. In contrast, the techniques disclosed herein accept and use data in different formats (e.g., accept data in ADT/HL-7 standard), enabling real-time, direct communication between the data source and the system that performs the analysis through a secure message upon admission/discharge. The techniques disclosed herein are thus far less time, labor, and resource intensive, and less prone to error. From a technical standpoint, the disclosed techniques save processing time and computing power. From a patient care standpoint, the disclosed techniques are a safer and more efficient way to process data.

The patient-centered techniques described herein are advantageously inclusive of all factors that may be relevant in readmission determinations. Further, the techniques lead to reduction in costs (e.g., healthcare costs, energy costs, etc.) and hospital readmissions, at least partially due to the use of real-time data. For example, conventional methods use data that is typically only available for review (e.g., manual review) after a certain time period (e.g., about 3 months), well after admissions and/or readmissions have occurred. Further, conventional methods use data that is pulled from numerous claims, e.g., insurance claims, which often increases the likelihood of inaccurate data (e.g., missing data, incorrect data, etc.) and/or increases the energy cost of data (e.g., requiring high levels of computer processing as well as manual processing, and increased use of computing resources). Unlike the conventional methods, the techniques described herein advantageously utilize data that is immediately available for real-time readmission analysis, increase accuracy of downstream determinations, and reduce computing and other energy-related costs.

The techniques described herein improve the ability of medical providers, insurance companies, accountable care organizations (ACOs), and patients to make real-time, evidence-based, actionable changes, e.g., to discharge protocols, which improves patient care and reduces the chances of dangerous and/or expensive future readmissions. The use of real-time data is especially advantageous in reducing readmissions, as each readmission negatively impacts patients and the financial performance of hospitals, insurance companies, ACOs, etc. Utilization of the techniques described herein, e.g., use of up-to-date, patient-centric information, enables determination of improved measures for reducing readmissions and/or improving quality of patient care. In addition to the benefits described herein, a person of ordinary skill in the arts would recognize that further technical advantages are apparent.

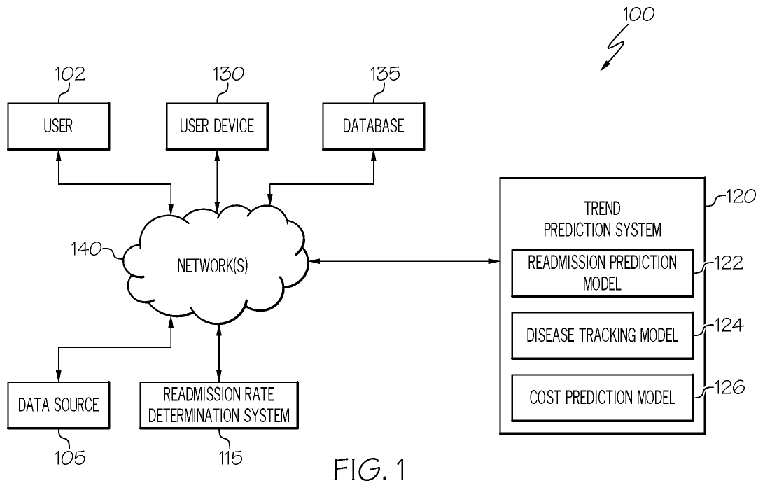

To address these challenges, FIG. 1 depicts an example environment for determining hospital readmission rates, according to some embodiments of the disclosure. Environment 100 of FIG. 1 depicts a data source 105, a readmission rate determination system 115, a trend prediction system 120, a user device 130, a user 102 that interacts with the user device 130, a database 135, and/or at least one network 140. In some techniques, database 135 includes an organizational repository.

Data source 105 is configured to obtain hospital admission data from one or more sources, such as from other aspects of environment 100, e.g., database 135, etc., and/or from third-party sources, e.g., servers and/or databases associated with hospitals, ACOs, insurance companies, etc. In some techniques, the hospital admission data is ingested and standardized. The hospital admission data may be received by other components of environment 100 (e.g., the readmission rate determination system 115, etc.) as a message/notification in HL7 or Admit, Discharge, and Transfer (ADT) format. The hospital admission data is associated with a plurality of individuals and includes at least one of patient identifier (e.g., medical record number (MRN)), admission and/or readmission data, discharge data, diagnosis data (e.g., a patient's admitting diagnosis, readmission rates associated with an admitting diagnosis, etc.), treatment data, patient demographic data (e.g., date of birth, age, residence data, etc.), patient social determinants (e.g., whether the patient is socially isolated or has next of kin as caretakers, etc.), hospital demographic data, insurance data, authorization data (e.g., insurance prior authorization, etc.), critical care document (CCD) summary data, admission, discharge, transfer (ADT) data, health level seven (HL7) messaging data, insurance claims data, disease indicators (e.g., International Classification of Diseases and Related Health Problems (ICD-10, ICD-11, etc.) codes, Systematized Nomenclature of Medicine—Clinical Terms (SNOMED), etc.), at least one admission date, readmission date(s), etc. The admission and/or readmission data may include patient symptoms or vital signs of presentation, patient admitting diagnosis, patient medical diagnoses, patient surgical diagnoses, patient medications, patient allergies, patient family history, patient social history, etc. The hospital admission data is arranged such that different types of data pertaining to each patient can be identified by the patient identifier that corresponds to the patient.

The hospital admission data may be obtained based on permission(s) provided by a patient. For example, data source 105 may obtain prior admission and readmission data from an ACO and diagnosis data from the admitting hospital to determine hospital readmission rates and/or trends, as discussed in further detail below. Data source 105 is configured to transmit data to other aspects of environment 100, e.g., to readmission rate determination system 115, trend prediction system 120, user device 130, database 135, etc.

Readmission rate determination system 115 is configured to determine a number of admissions and associated readmissions, and a readmission rate, in at least one time frame of interest. Readmission rate determination system 115 is configured to receive hospital admission data, e.g., at least one indicator of a disease of interest and at least one admission date, from one or more aspects of environment 100, e.g., data source 105, trend prediction system 120, user device 130, database 135, etc.

Readmission rate determination system 115 is configured to determine a number of admissions and readmissions. In some techniques, readmission rate determination system 115 is configured to determine a number of admissions and readmissions for a time frame of interest. The time frame of interest is any suitable value, e.g., 10 days, 20 days, 30 days, 45 days, 60 days, 90 days, etc. It should be understood that other values not discussed herein are also contemplated values for the time frame of interest. For example, if a primary admission occurs on January 1 and the time frame of interest is 30 days, readmissions that occur January 13 and January 25 are included, while a readmission that occurs on February 17 is excluded.

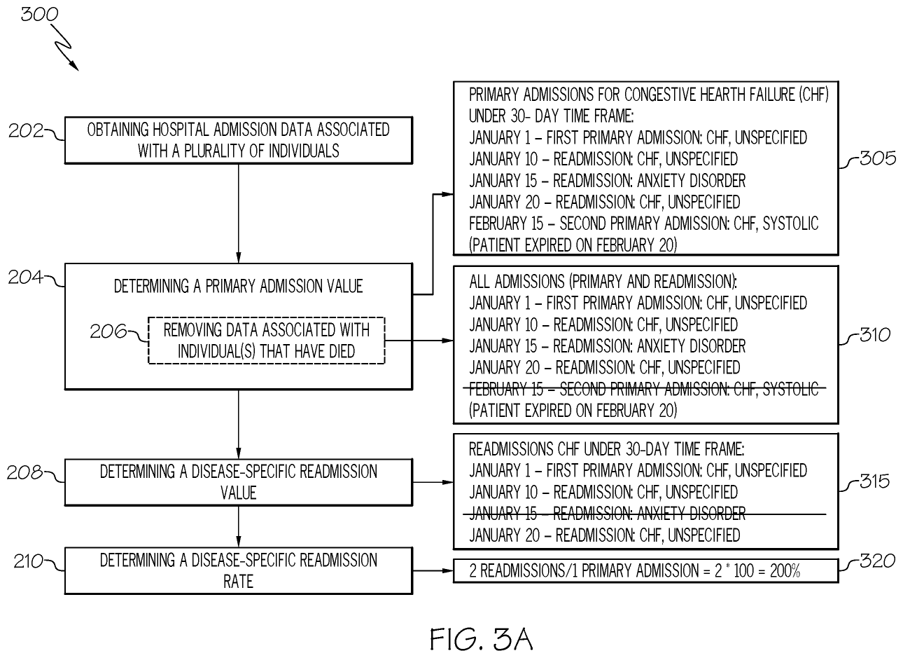

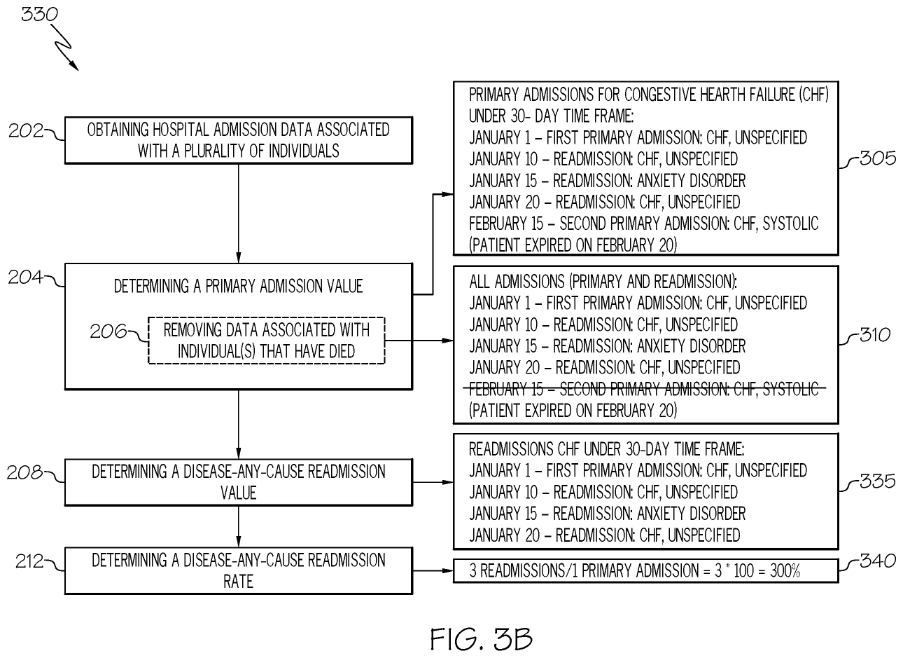

As discussed in more detail below, in determining the number of admissions and readmissions, readmission rate determination system 115 may be disease-specific, disease-agnostic (see FIGS. 3A and 3B, respectively), or both. Continuing the previous example, the number of admissions and readmissions may be determined for a given disease (e.g., heart disease) for a time frame of interest (e.g., 45 days), or may be determined to be disease-agnostic for the time frame of interest. Readmission rate determination system 115 is configured to determine a readmission rate (e.g., a disease-specific readmission rate and/or a disease-agnostic readmission rate) based on the determined number of admissions and readmissions in a given time frame of interest. An example method for determining a readmission rate is described in further detail below.

Readmission rate determination system 115 transmits the number of admissions and readmissions, the readmission rate, the time frame of interest, etc. to other aspects of environment 100, e.g., trend prediction system 120, user device 130, database 135, etc.

Trend prediction system 120 is configured to determine at least one trend. In some techniques, trend prediction system 120 utilizes at least one machine learning model to generate the at least one trend. Trend prediction system 120 is configured to use at least one trained machine learning model, e.g., a trained trend prediction machine learning model, to determine at least one trend. An example method for training and/or using the trained trend prediction machine learning model is described in more detail below.

In one embodiment, the trained trend prediction machine learning model is configured for unsupervised machine learning that does not require training using known outcomes, e.g., correct responses. Unsupervised machine learning utilizes machine learning algorithms to analyze and cluster unlabeled datasets and discover hidden patterns or data groupings, e.g., similarities and differences within data, without supervision. In one example embodiment, the unsupervised machine learning implements approaches that includes clustering (e.g., deep embedded clustering, K-means clustering, hierarchical clustering, probabilistic clustering), association rules, classification, principal component analysis (PCA), or the like. The trained trend prediction machine learning model utilizes the unsupervised machine learning techniques to determine at least one trend.

In one embodiment, the trained trend prediction machine learning model is configured for supervised machine learning that utilizes training data, e.g., admission and readmission values, readmission rates, target readmission rates, and actual readmission rates, etc., for training a machine learning model configured to determine at least one trend. In one example embodiment, the trained trend prediction machine learning model performs model training using training data that contains input and correct output (e.g., labels), to allow the model to learn over time. The training is performed based on the deviation of a processed result from a documented result when the inputs are fed into the machine learning model, e.g., an algorithm measures its accuracy through the loss function, adjusting until the error has been sufficiently minimized. In one embodiment, the trained trend prediction machine learning model randomizes the ordering of the training data, visualizes the training data to identify relevant relationships between different variables, identifies any data imbalances, and splits the training data into two parts where one part is for training a model and the other part is for validating the trained model, de-duplicating, normalizing, correcting errors in the training data, and so on. The trained trend prediction machine learning model implements various machine learning techniques, e.g., K-nearest neighbors, cox proportional hazards model, random forest model, decision tree learning, association rule learning, neural network (e.g., recurrent neural networks, graph convolutional neural networks, deep neural networks, autoencoders), inductive programming logic, support vector machines (SVM), Bayesian models, Gradient boosted machines (GBM), LightGBM (LGBM), Xtra tree classifier, etc.

In one embodiment, the trained trend prediction machine learning model implements natural language processing (NLP) to analyze, understand, and derive meaning from at least one data entry, e.g., readmission notes from a treating medical professional, electronic medical records, etc. NLP is applied to analyze text, allowing machines to understand how humans speak/write, enabling real-world applications such as automatic text summarization, sentiment analysis, topic extraction, named entity recognition, parts-of-speech/text tagging, relationship extraction, stemming, and/or the like. In one embodiment, NLP generally encompasses techniques including, but not limited to, keyword search, finding relationships (e.g., synonyms, hypernyms, hyponyms, and meronyms), extracting information (e.g., keywords, key phrases, search terms), classifying, and determining positive/negative sentiment of documents. In one example embodiment, the trained trend prediction machine learning model utilizes NLP to perform text summarization on data to determine relevant medical data that may not have otherwise been obtained via the disease indicators, e.g., ICD-10 codes.

In some techniques, trend prediction system includes at least a readmission prediction model 122, a disease tracking model 124, a cost prediction model 126, etc. Readmission prediction model 122 is configured to predict future admissions and readmissions. Readmission prediction model 122 may receive admission and/or readmission data, e.g., admission data, readmission data, readmission rates, etc. from one or more aspects of environment 100, e.g., data source 105, readmission rate determination system 115, other aspects of trend prediction system 120, user device 130 (e.g., via user 102 interact with user device 130), database 135, etc. Readmission prediction model 122 may use a trained machine learning model, e.g., the trained trend prediction machine learning model and/or a trained readmission prediction machine learning model, to predict future admissions and readmissions. In some techniques, readmission prediction model 122 includes a readmission prediction machine learning model that is trained to predict readmission.

Disease tracking model 124 is configured to track at least one disease, at least one disease stage, etc., e.g., based at least on the readmission data. For example, disease tracking model 124 may track CHF, kidney disease, the stage(s) of kidney disease, etc. Disease tracking model 124 may receive disease-related data, e.g., diagnoses, treatment plans, etc. from one or more aspects of environment 100, e.g., data source 105, readmission rate determination system 115, other aspects of trend prediction system 120, user device 130 (e.g., via user 102 interact with user device 130), database 135, etc. Disease tracking model 124 may use a trained machine learning model, e.g., the trained trend prediction machine learning model and/or a trained disease tracking machine learning model, to predict disease progression. In some techniques, disease tracking model 124 includes a disease tracking machine learning model that is trained to predict disease progression, remission, comorbid diagnoses, etc.

Cost prediction model 126 is configured to predict costs associated with past, present, and/or future admissions and readmissions. Cost prediction model 126 may receive cost-related data, e.g., short-term and/or long-term treatment costs, prescription costs, outpatient costs, other medical costs, etc. from one or more aspects of environment 100, e.g., data source 105, readmission rate determination system 115, other aspects of trend prediction system 120, user device 130 (e.g., via user 102 interact with user device 130), database 135, etc. Cost prediction model 126 may use a trained machine learning model, e.g., the trained trend prediction machine learning model and/or a trained cost prediction machine learning model, to predict costs. In some techniques, cost prediction model 126 includes a cost prediction machine learning model that is trained to predict costs associated with current and/or future treatment(s), prescription costs, outpatient costs (e.g., physical therapy), etc.

Trend prediction system 120 (and/or each of readmission prediction model 122, disease tracking model 124, cost prediction model 126, etc.) transmits the predicted trends, e.g., the predicted readmission(s), disease prediction(s), cost prediction(s), etc., to other aspects of environment 100, e.g., readmission rate determination system 115, user device 130, database 135, etc.

User device 130 is any electronic device, e.g., a cellular phone, a tablet, a personal computer, a wearable device, Internet of Things (IoT) device, any suitable device, etc. User device 130 is configured to obtain data from any suitable aspect of environment 100, such as data source 105, admission determination system 110, readmission rate determination system 115, trend prediction system 120, database 135, other devices (e.g., IoT device) in the environment 100, etc. User device 130 hosts one or more applications, such as a readmission application, that is capable of collecting, storing, and/or transmitting user data for determining hospital readmission rates and/or probabilities, as described in further detail below. For example, user 102 (e.g., patient, medical provider, etc.) receives hospital readmission data and/or probabilities via user device 130.

One or more of the components in FIG. 1 may communicate with each other and/or other systems, e.g., across the at least one network 140. In some embodiments, the at least one network 140 may connect one or more components of environment 100 via a wired connection, e.g., a USB connection between user device 130 and trend prediction system 120. In some embodiments, the at least one network 140 may connect one or more aspects of environment 100 via an electronic network connection, for example a wide area network (WAN), a local area network (LAN), personal area network (PAN), or the like. In some embodiments, the electronic network connection includes the internet, and information and data provided between various systems occurs online. “Online” may mean connecting to or accessing source data or information from a location remote from other devices or networks coupled to the Internet. Alternatively, “online” may refer to connecting or accessing an electronic network (wired or wireless) via a mobile communications network or device. The Internet is a worldwide system of computer networks—a network of networks in which a party at one computer or other device connected to the network may obtain information from any other computer and communicate with parties of other computers or devices. The most widely used part of the Internet is the World Wide Web (often-abbreviated “WWW” or called “the Web”). A “website page,” a “portal,” or the like generally encompasses a location, data store, or the like that is, for example, hosted and/or operated by a computer system so as to be accessible online, and that may include data configured to cause a program such as a web browser to perform operations such as send, receive, or process data, generate a visual display, e.g., a window, and/or an interactive interface, or the like. In any case, the connections within the environment 100 are network, wired, any other suitable connection, or any combination thereof.

Although depicted as separate components in FIG. 1, it should be understood that a component or portion of a component in the environment 100 may, in some embodiments, be integrated with or incorporated into one or more other components. For example, all or a portion of readmission rate determination system 115 is integrated into trend prediction system 120 or the like. In another example, data source 105 is integrated into database 135. In another example, readmission prediction model 122, disease tracking model 124, and cost prediction model 126 are distributed across one or more systems. In some embodiments, operations or aspects of one or more of the components discussed above are distributed amongst one or more other components. Any suitable arrangement and/or integration of the various systems and devices of the environment 100 is used.

In some techniques, the methods described below include analyzing data, e.g., hospital admission data, that has been organized into one or more datasets. Steps 202-216 may therefore be implemented using the one or more datasets. For example, at least one hospital admission dataset may be generated, e.g., as used in step 202. The at least one hospital admission dataset includes hospital admission data. For example, a first hospital admission dataset may include a patient's MRN, ADT data, and residence data, and a second hospital admission dataset may include hospital demographic data, insurance data, and HL7 messaging data. Any combination of data may be used to generate the at least one hospital admission dataset and the data included in generating the at least one hospital admission dataset may be customized by a user, e.g., user 102.

A raw disease-specific dataset is generated from the hospital admission dataset. The raw disease-specific dataset includes at least one disease indicator and a subset of further data from the at least one hospital admission dataset, e.g., a patient identifier, patient demographic information (e.g., date of birth, insurance type(s), etc.), hospital demographic information (e.g., state, count, city, etc.), ADT data, etc. The raw disease-specific dataset includes multiple rows of data from a single patient organized based on the episode of care (e.g., based on an admission). In some techniques, data that may cause duplicative entries are removed or ignored. For example, if a patient has dual insurance, the raw disease-specific dataset identifies or flags the double insurance coverage to avoid a double entry that can skew the analysis. In some embodiments, the raw disease-specific dataset is organized based on at least one criteria, e.g., MRN, admission data, discharge data, patient location, hospital location, etc. For example, the raw disease-specific dataset is organized based on hospital locations in Las Vegas, Nevada. In another example, the raw disease-specific dataset is organized based on patient discharge dates in the last 12 months. The dataset is organized based on any length of time, e.g., 6 months, 12 months, 2 years, etc. depending on the readmission quality improvement goals.

In some techniques, certain data is removed from the raw disease-specific dataset. For example, patients with a discharge status of expired or died in the hospital are removed. The data removed from the raw disease-specific dataset may be within a length of time, e.g., the patient's death is within 30 days of discharge. In some techniques, the rows of data removed based on the patient's death are saved as at least one expiration dataset and/or saved to a database, e.g., database 135. For example, data removed based on the patient's death within 30 days of discharge is stored as an expiration dataset. The data remaining in the raw disease-specific dataset after the expiration data is removed and/or stored separately to a database, e.g., database 135, as a modified raw disease-specific dataset.

In some techniques, a raw disease-any-cause dataset is generated. Similar to the techniques discussed above, the raw disease-any-cause dataset is generated from the hospital admission dataset. The rows of the disease-any-cause dataset are not ignored and/or removed based on a disease indicator. In some techniques, the disease-any-cause dataset is generated by adding the expiration dataset back into the disease-specific dataset. The rows of the disease-any-cause dataset are organized and/or analyzed using techniques similar to those discussed above, e.g., based on MRN, admission data, to prevent duplications, etc. Similar to above, data indicating a patient expiration, e.g., within 30 days of discharge, is removed and/or stored in a database. The data remaining in the raw disease-any-cause dataset after the expiration data is removed and/or stored separately to a database, e.g., database 135, as a modified raw disease-any-cause dataset.

The modified raw disease-specific dataset and/or a modified raw disease-any-cause dataset may be organized based on at least one feature, e.g., a primary feature, a secondary feature, a tertiary feature, etc. For example, the rows of data in the modified raw disease-specific dataset are organized based on MRNs as the primary feature and discharge date as the secondary feature. In another example, the rows of data in the modified raw disease-any-cause dataset are organized based on patient age (e.g., over 65 years old or under 65 years old) as the primary feature and discharge date at the secondary feature. In one embodiment, the organized rows are analyzed based on one or more of the at least one feature, e.g., based on the MRN, based on the discharge date, based on both the MRN and discharge date, etc. In some techniques, the rows of the modified raw disease-specific dataset are organized based on a disease indicator. For example, rows that share a common disease indicator, e.g., at least one disease indicator for CHF, are retained in modified raw disease-specific dataset, and rows that do not contain the common disease indicator are ignored and/or removed. The rows of modified raw disease-specific dataset remaining after the rows that do not contain the common disease indicator are removed are saved as a disease-specific dataset. The removed data of the rows that do not contain the common disease indicator is stored in a database, e.g., in database 135, as a disinterest data set.

As discussed in more detail below, the modified raw disease-specific dataset and/or the modified raw disease-any-cause dataset are analyzed to determine a number of primary admissions and/or a number of readmissions for each dataset.

From the number of disease-specific primary admissions and/or the number of disease-specific readmissions, a disease-specific primary admission value and/or a disease-specific readmission value are determined. From the disease-specific primary admission value and the disease-specific readmission value, a disease-specific readmission rate is determined.

From the number of disease-any-cause primary admissions and/or the number of disease-any-cause readmissions, a disease-any-cause primary admission value and/or a disease-any-cause readmission value are determined. From the disease-any-cause primary admission value and the disease-any-cause readmission value, a disease-any-cause readmission rate is determined.

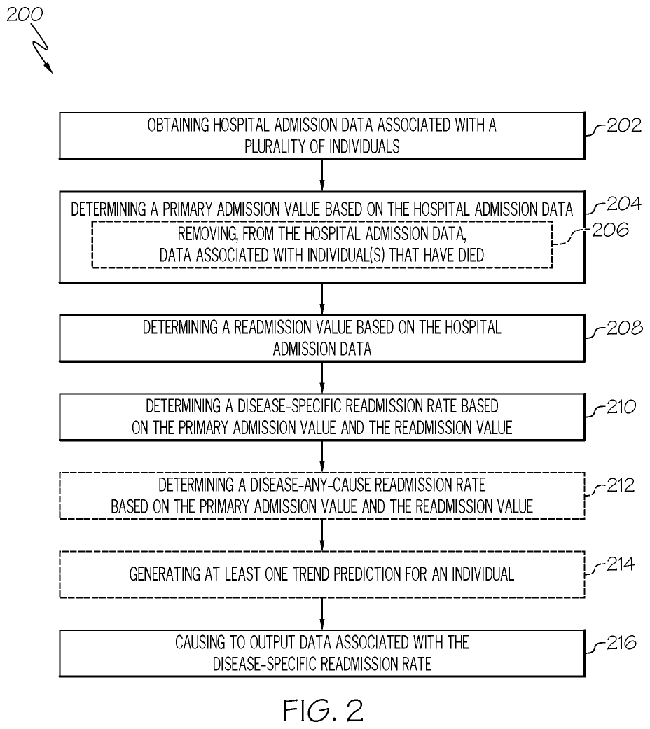

In the following steps depicted in FIGS. 2, 3A, and 3B, one or more of the datasets described above may be used. FIG. 2 depicts an example computer-implemented method for determining hospital readmission rates, according to some embodiments of the disclosure. In one embodiment, method 200 is performed by one or more components depicted in FIG. 1. In some embodiments, method 200 is performed by one or more servers or computing devices implementing the one or more components depicted in FIG. 1.

At step 202, data associated with a plurality of individuals is obtained, e.g., hospital admission data, the hospital admission dataset, etc. As described herein, the hospital admission data is associated with a plurality of individuals and includes at least one indicator of a disease of interest, e.g., congestive heart failure (CHF), and at least one admission date for each individual. As discussed above in reference to FIG. 1, for example, the hospital admission data is arranged such that different types of data pertaining to each patient can be identified by the patient identifier that corresponds to the patient. Therefore, using the hospital admission data, a readmission rate for a specific patient can be calculated, as well as a readmission rate across a patient population associated with a healthcare service provider or an organization.

At step 204, a primary admission value is determined based at least on the hospital admission data. The primary admission value includes a total number of primary admissions in a time period that may be predefined (e.g., from a first date to a second date, also referred to herein as a “second time frame of interest”). Primary admissions are determined using the hospital admission data, and by setting a first time frame of interest by which admissions in the hospital admission data during the predefined time period can be analyzed (e.g., a first time window that is applied to the hospital admission data on a rolling basis). In some techniques, the first time frame of interest is a time frame beginning at a primary admission. In one embodiment, one or more primary admissions, among a plurality of admissions in the hospital admission data, are identified by setting a first time frame of interest within which a primary admission is present. For example, in the case that the hospital admission data identifies multiple admissions from January 1 through February 28, and a first time frame of interest is set to be 31 days, the first admission that took place during the first 31-day period starting from January 1 (e.g., January 1 through January 31) is determined to be a primary admission for that first 31-day period, and the first admission that took place during the second 31-day period starting from February 1 (e.g., February 1 through March 3) is determined to be a primary admission for that second 31-day period. Therefore, a primary admission may be a first admission in each of the iterative sets of time frames of interest. Each of the at least one first time frame of interest has zero or one primary admission. All other admissions in each time period set by the first time frame of interest are considered readmissions, as discussed in further detail below.

For illustrative purposes, FIGS. 3A and 3B depict example methods for determining hospital readmission rates. Specifically, step 305 depicts an example of how a primary admission value is determined. As depicted in step 305, the hospital admission data identifies multiple admissions that took place during a time period, e.g., from January 1 through February 15, and a first time frame of interest has been set to be 30 days (e.g., “under 30-day time frame”). In the first 30-day period, the admission that took place on January 1 is determined to be a primary admission (e.g., “first primary admission”) and all other admissions during that period are considered readmissions. With continuing reference to step 305, in the second 30-day period, the admission that took place on February 15 is determined to be a primary admission (e.g., “second primary admission”). Considering step 305 alone, the primary admission value is 2, as the total number of primary admissions identified from the hospital admission data for the time period (e.g., from January 1 through February 15) is 2 (e.g., the admissions on January 1 and February 15). However, as discussed below with reference to step 206 and related step 310, the primary admission value may further be adjusted under certain circumstances.

Optionally, at step 206, data associated with any of the plurality of individuals that have died are removed from the hospital admission data. While this data may be removed from the hospital admission data, which is considered for the purpose of determining the readmission rate, the removed data may still be retained elsewhere in the system and be used to determine other important variables or insights, e.g., as an expiration dataset. In some techniques, the expiration dataset may be used downstream, e.g., to determine mortality rates per hospital. In some techniques, the removed data is stored as a dataset, e.g., in database 135, with the patient's date of death and/or patient identification number, e.g., MRN. The admissions removed based on patient expiration (e.g., death) are determined based on a patient expiration time frame of interest. The patient expiration time frame of interest is a time frame in which, if a patient dies, any admission and/or readmission date in that time frame is removed from the hospital admission data. For example, as depicted in step 310 of FIGS. 3A and 3B, for a 30-day first time frame of interest and 30-day patient expiration time frame of interest, a patient has a first primary admission on January 1, a second primary admission on February 15, and the patient died on February 20. Since the second primary admission on February 15 falls within the 30-day patient expiration time frame of interest from the patient expiration date of February 20, the second primary admission (and associated readmissions, if any) is removed from the data. Because the second primary admission is removed from consideration, the primary admission value is 1, as the total number of primary admissions identified from the hospital admission data is 1. In another example, for a 30-day first time frame of interest and 30-day patient expiration time frame of interest, if a patient has a first primary admission on January 1, a second primary admission on February 15, a third primary admission on March 7, and the patient died on March 10, the third primary admission (and associated readmissions, if any) is removed from the data. Thus, the primary admission value (as well as the readmission value or a total number of readmissions) may be adjusted based on additional considerations and factors, such as patient death that occurs within a set time from an admission. As discussed herein, any time frame of interest may be implemented. For example, the first time frame of interest and the patient expiration time frame of interest are not limited to 30 days as illustrated in the figures—instead, the first time frame of interest may be 60 days and the patient expiration time frame of interest may be 45 days.

At step 208, a readmission value is determined based on the hospital admission data. The readmission value includes a total number of readmissions associated with a primary admission during a time period that may be predefined. A readmission is determined in relation to a primary admission in the first time period of interest, e.g., the first time period of interest associated with determining the at least one primary admission at step 204. For example, in the case that the hospital admission data identifies multiple admissions from January 1 through February 28, and a first time frame of interest is set to be 31 days, the first admission that took place during the first 31-day period starting from January 1 (e.g., January 1 through January 31) is determined to be a primary admission for that first 31-day period and any subsequent admission until January 31 is a readmission, and the first admission that took place during the second 31-day period starting from February 1 (e.g., February 1 through March 3) is determined to be a primary admission for that second 31-day period and any subsequent admission until March 3 is a readmission. Therefore, readmissions are defined in relation to a primary admission in a first time frame of interest.

A readmission and/or the readmission value may be disease-specific (e.g., based on one indicator of a disease of interest, such as an ICD-10 code) or disease-agnostic (e.g., based on more than one indicator of a disease of interest, such as an ICD-10 code). For example, as depicted in step 315 of FIG. 3A, if the time frame of interest is 30 days, the readmissions associated with the first primary admission (January 1) are the January 10, January 15, and January 20 readmissions. To determine the disease-specific readmission value, any readmissions not associated with the at least one indicator of a disease of interest, e.g., not associated with CHF, are removed. As shown in step 315, the January 15 readmission is removed as it is related to a readmission for an anxiety disorder, not for CHF. Therefore, in this example, the disease-specific readmission value is 2. In some techniques, more than one disease may be considered in determining the disease-specific readmission value. For example, if a patient has admissions and/or readmissions related to 5 different diseases, the disease-specific readmission value is determined based on one, two, three, four, or five of the diseases.

To determine the disease-any-cause readmission value, or the disease-agnostic readmission value, as depicted in step 335 of FIG. 3B, all readmissions are considered. In some techniques, the disease-any-cause readmission value is determined by adding back in (or simply including) values previously removed in determining the disease-specific readmission value. For example, the disease-any-cause readmission value may be determined by adding back the readmission that was removed in step 315 (January 15). Therefore, in the example depicted by step 335, the disease-any-cause readmission value is 3.

At step 210, a disease-specific readmission rate is determined for a time period that may be predefined (e.g., a second time frame of interest). In determining the disease-specific readmission rate, the readmission value and the primary admission value share a common indicator of a disease of interest, e.g., share an ICD-10 code. The time period set for step 210 may be the same as, or different from, the time frame of interest for step 204 and/or step 208. For example, the time frame of interest used at step 208 may be 30 days and the time period used at step 210 may be one year. Any time frame and/or combination of time frames are contemplated for the one or more time frames discussed herein.

In some techniques, the disease-specific readmission rate is determined by calculating a percentage based on a ratio between the disease-specific readmission value and the primary admission value. As depicted by step 320 of FIG. 3A, the disease-specific readmission rate for 2 readmissions and 1 primary admission is 200% (e.g., the readmission value “2” divided by the primary admission value “1,” times 100). In some techniques, the disease-specific readmission rate is determined by using a trained disease-specific readmission rate machine learning model. The trained disease-specific readmission rate machine learning model is trained by receiving disease-specific readmission rate training data and training a machine learning model to infer a readmission rate. The disease-specific readmission rate training data includes at least a portion of hospital admission data, including a plurality of admission dates associated with a plurality of users and a plurality of indicators of diseases of interest corresponding to the plurality of admission dates, etc.

Optionally, at step 212, a disease-any-cause readmission rate is determined for a time period that may be predefined (e.g., a second time frame of interest). In determining the disease-any-cause readmission rate, the readmission value and the primary admission value can be associated with any number of indicators of diseases of interest, e.g., the readmission value and the primary admission value may have at least one different ICD-10 code. The time period set for step 212 may be the same as, or different from, the time frame of interest for steps 204 and/or 208. For example, the time frame of interest set for step 208 may be 30 days and the time period set for step 212 may be one year. Any time frame and/or combination of time frames are contemplated for the one or more time frames discussed herein.

In some techniques, the disease-any-cause readmission rate is determined by calculating a percentage based on a ratio between the disease-any-cause readmission value and the primary admission value. As depicted by step 340 of FIG. 3B, the disease-any-cause readmission rate for 3 readmissions and 1 primary admission is 300% (e.g., the readmission value “3” divided by the primary admission value “1,” times 100). In some techniques, the disease-any-cause readmission rate is determined by using a trained disease-agnostic readmission rate machine learning model. The trained disease-agnostic readmission rate machine learning model is trained by receiving disease-agnostic readmission rate training data and training a machine learning model to infer a readmission rate. The disease-agnostic readmission rate training data includes at least a portion of hospital admission data, including a plurality of admission dates associated with a plurality of users and a plurality of indicators of diseases of interest corresponding to the plurality of admission dates, etc.

Optionally, at step 214, at least one trend prediction is generated for an individual. The at least one trend prediction includes at least one of readmission prediction(s), disease tracking prediction(s), cost prediction(s), etc. The at least one trend prediction may be generated based on at least one of hospital admission data, primary admission value, readmission value, disease-specific readmission rate, disease-any-cause readmission rate, etc.

In some embodiments, the at least one trend prediction is determined via at least one trained trend prediction machine learning model, e.g., a trained readmission prediction machine learning model, a trained disease tracking machine learning model, a trained cost prediction machine learning model, etc. For example, at least hospital data are compared to disease-specific readmission rate (e.g., as determined by step 210 of FIG. 3A) to determine at least a readmission prediction. The trained readmission prediction machine learning model is trained by receiving readmission data and training a machine learning model to infer whether a user is likely to be readmitted (e.g., to determine a probability that the user is readmitted). The readmission prediction training data includes at least one of disease codes, primary admissions and associated readmissions, disease prognosis, lifestyle factors, readmission cost data, etc.

In some techniques, the trained readmission prediction machine learning model, e.g., a Support Vector Model (SVM), may be a decision tree configured to predict hospital readmissions by analyzing data and identifying factors that are associated with higher or lower rates of readmission. For example, trained readmission prediction machine learning model may be trained on at least the readmission value, the primary admission value, and the at least one common indicator of a disease of interest to predict whether or not the patient will be readmitted to the hospital within a certain time period, e.g., 30 days. The trained readmission prediction machine learning model also analyzes the data, e.g., the admission value, the readmission value, the at least one common indicator of a disease of interest, hospital admission data, etc., and identifies which features are most predictive of readmission. These features may include factors such as age, length of stay, hospital of admission, presence of comorbidities, previous history of readmissions, etc. Once the trained readmission prediction machine learning model had learned the training dataset and was validated on test data, it could then be used to predict whether patients are at risk of readmission based on their individual characteristics. For example, if a patient were identified as being at high risk for readmission based on the result of the trained readmission prediction machine learning model, then healthcare providers could proactively take steps to prevent future readmissions. Such measures include increased follow-up appointments, sooner appointments, early involvement of a case manager, deployment of post-acute resources, e.g., a home care nurse, etc.

In another example, a patient may be admitted to a hospital and receive a diagnosis of a malignant neoplasm of the liver. Diagnoses such as malignant neoplasm of the liver, CHF, etc. typically have a higher readmission rate compared to diagnoses such as laceration of the finger, broken humerus bone, etc. Thus, the trained readmission prediction machine learning model may assign more weight to certain types of admissions in determining the likelihood of readmission.

In another example, at least hospital data are compared to disease-specific readmission rate (e.g., as determined by step 210 of FIG. 3A) to determine at least a disease tracking prediction. The disease tracking prediction includes at least one of disease prognosis prediction, disease progression prediction, etc. The trained disease tracking machine learning model is trained by receiving readmission data and training a machine learning model to infer at least one of disease prognosis, disease comorbidity, recovery time, etc. The disease tracking training data includes at least one of disease codes, primary admissions and associated readmissions, disease prognosis, lifestyle factors, disease comorbidities, etc.

In another example, at least hospital data are compared to disease-specific readmission rate (e.g., as determined by step 210 of FIG. 3A) to determine at least a cost prediction. The trained cost prediction machine learning model is trained by receiving readmission data and training a machine learning model to infer what costs are expected to be incurred in current and/or future admissions and/or readmissions. The cost prediction training data includes at least one of disease codes, primary admissions and associated readmissions, disease prognosis, lifestyle factors, cost of care estimates, prescription drugs costs, patient insurance data, etc.

At step 216, data associated with the disease-specific readmission rate is caused to be outputted, e.g., to a GUI associated with user device 130. Also, if a trend prediction for an individual has been generated as discussed with reference to step 214, data associated with the trend prediction may also be outputted to the GUI associated with user device 130. Also, if a disease-any-cause readmission rate has been determined as discussed with reference to step 212, data associated with the disease-any-cause readmission rate is also caused to be outputted to the GUI associated with user device 130. In other words, the readmission rate(s) and any trend prediction determined during the data process flow are provided to the user via the GUI of the user device 130.

In addition to displaying the determined data, the readmission rate determination system 115 and/or the trend prediction system 120 can use the determined data to take proactive actions to prevent complications associated with readmissions. For example, if the determined readmission rate is equal to or higher than a predefined threshold, the system can provide alerts or other helpful information (e.g., recommendations) that may aid healthcare providers to proactively take steps to prevent future readmissions. Such measures include increased follow-up appointments, sooner appointments, early involvement of a case manager, deployment of post-acute resources, e.g., a home care nurse, etc.

Additionally, the determined data can be used to monitor patient populations, certain hospitals, certain regions, etc. For example, if there were substantial readmissions for patients admitted to a particular hospital, the determined data may be used to determine a potential cause. In another example, if there was a reduction in the readmission rate following the implementation of a program, e.g., a medical education pilot program at a hospital, the determined data as a result of the program may be used to determine patient outcomes, improve patient experience, and save on hospitalization costs, e.g., to extrapolate the results to other hospitals.

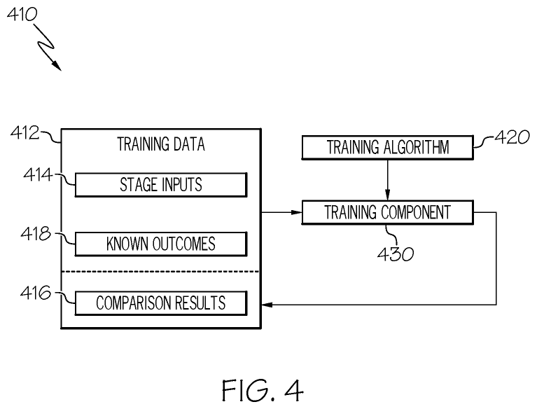

One or more implementations disclosed herein include and/or are implemented using a machine learning model, e.g., the trained readmission prediction machine learning model, the trained disease tracking machine learning model, the trained cost prediction machine learning model. For example, one or more of the engines of trend prediction system 120 are implemented using a machine learning model and/or are used to train the machine learning model. A given machine learning model is trained using the training flow chart 400 of FIG. 4. The training data 412 includes one or more of stage inputs 414 and the known outcomes 418 related to the machine learning model to be trained. The stage inputs 414 are from any applicable source including text, visual representations, data, values, comparisons, and stage outputs, e.g., one or more outputs from one or more steps from FIG. 2. The known outcomes 418 are included for the machine learning models generated based on supervised or semi-supervised training. An unsupervised machine learning model is not trained using the known outcomes 418. The known outcomes 418 includes known or desired outputs for future inputs similar to or in the same category as the stage inputs 414 that do not have corresponding known outputs.

The training data 412 and a training algorithm 420, e.g., one or more of the modules implemented using the machine learning model and/or are used to train the machine learning model, is provided to a training component 430 that applies the training data 412 to the training algorithm 420 to generate the machine learning model. According to an implementation, the training component 430 is provided comparison results 416 that compare a previous output of the corresponding machine learning model to apply the previous result to re-train the machine learning model. The comparison results 416 are used by the training component 430 to update the corresponding machine learning model. The training algorithm 420 utilizes machine learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, classifiers such as K-Nearest Neighbors, and/or discriminative models such as Decision Forests and maximum margin methods, the model specifically discussed herein, or the like.

The machine learning model used herein is trained and/or used by adjusting one or more weights and/or one or more layers of the machine learning model. For example, during training, a given weight is adjusted (e.g., increased, decreased, removed) based on training data or input data. Similarly, a layer is updated, added, or removed based on training data/and or input data. The resulting outputs are adjusted based on the adjusted weights and/or layers.

In general, any process or operation discussed in this disclosure is understood to be computer-implementable, such as the processes illustrated in FIGS. 2, 3A, and 3B, are performed by one or more processors of a computer system as described herein. A process or process step performed by one or more processors is also referred to as an operation. The one or more processors are configured to perform such processes by having access to instructions (e.g., software or computer-readable code) that, when executed by one or more processors, cause one or more processors to perform the processes. The instructions are stored in a memory of the computer system. A processor is a central processing unit (CPU), a graphics processing unit (GPU), or any suitable type of processing unit.

A computer system, such as a system or device implementing a process or operation in the examples above, includes one or more computing devices. One or more processors of a computer system are included in a single computing device or distributed among a plurality of computing devices. One or more processors of a computer system are connected to a data storage device. A memory of the computer system includes the respective memory of each computing device of the plurality of computing devices.

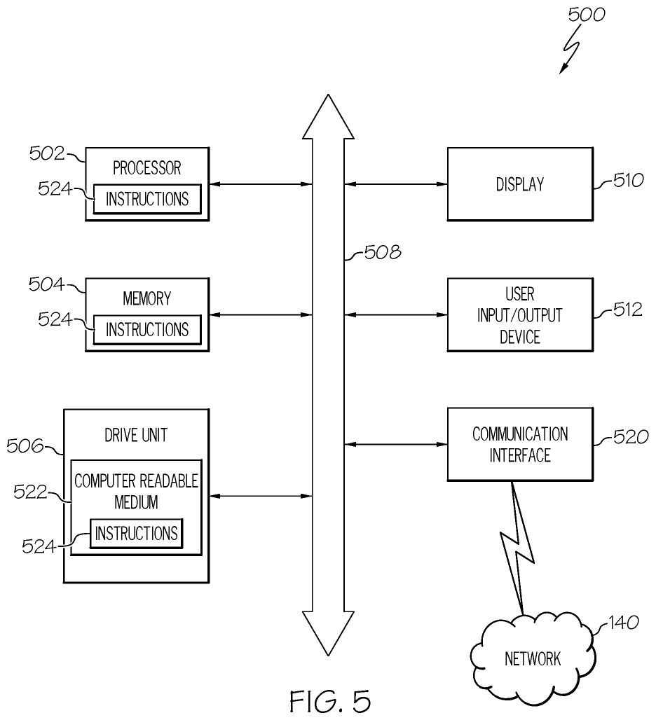

FIG. 5 illustrates an implementation of a computer system that executes techniques presented herein. The computer system 500 includes a set of instructions that are executed to cause the computer system 500 to perform any one or more of the methods or computer based functions disclosed herein. The computer system 500 operates as a standalone device or is connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” refers to any device or portion of a device that processes electronic data, e.g., from registers and/or memory to transform that electronic data into other electronic data that, e.g., is stored in registers and/or memory. A “computer,” a “computing machine,” a “computing platform,” a “computing device,” or a “server” includes one or more processors.

In a networked deployment, the computer system 500 operates in the capacity of a server or as a client user computer in a server-client user network environment, or as a peer computer system in a peer-to-peer (or distributed) network environment. The computer system 500 is also implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 500 is implemented using electronic devices that provide voice, video, or data communication. Further, while the computer system 500 is illustrated as a single system, the term “system” shall also be taken to include any collection of systems or sub-systems that individually or jointly execute a set, or multiple sets, of instructions to perform one or more computer functions.

As illustrated in FIG. 5, the computer system 500 includes a processor 502, e.g., a central processing unit (CPU), a graphics processing unit (GPU), or both. The processor 502 is a component in a variety of systems. For example, the processor 502 is part of a standard personal computer or a workstation. The processor 502 is one or more processors, digital signal processors, application specific integrated circuits, field programmable gate arrays, servers, networks, digital circuits, analog circuits, combinations thereof, or other now known or later developed devices for analyzing and processing data. The processor 502 implements a software program, such as code generated manually (i.e., programmed).

The computer system 500 includes a memory 504 that communicates via bus 508. The memory 504 is a main memory, a static memory, or a dynamic memory. The memory 504 includes, but is not limited to computer-readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 504 includes a cache or random-access memory for the processor 502. In alternative implementations, the memory 504 is separate from the processor 502, such as a cache memory of a processor, the system memory, or other memory. The memory 504 is an external storage device or database for storing data. Examples include a hard drive, compact disc (“CD”), digital video disc (“DVD”), memory card, memory stick, floppy disc, universal serial bus (“USB”) memory device, or any other device operative to store data. The memory 504 is operable to store instructions executable by the processor 502. The functions, acts, or tasks illustrated in the figures or described herein are performed by the processor 502 executing the instructions stored in the memory 504. The functions, acts, or tasks are independent of the particular type of instruction set, storage media, processor, or processing strategy and are performed by software, hardware, integrated circuits, firmware, micro-code, and the like, operating alone or in combination. Likewise, processing strategies include multiprocessing, multitasking, parallel processing, and the like.

As shown, the computer system 500 further includes a display 510, such as a liquid crystal display (LCD), an organic light emitting diode (OLED), a flat panel display, a solid-state display, a cathode ray tube (CRT), a projector, a printer or other now known or later developed display device for outputting determined information. The display 510 acts as an interface for the user to see the functioning of the processor 502, or specifically as an interface with the software stored in the memory 504 or in the drive unit 506.

Additionally or alternatively, the computer system 500 includes an input/output device 512 configured to allow a user to interact with any of the components of the computer system 500. The input/output device 512 is a number pad, a keyboard, a cursor control device, such as a mouse, a joystick, touch screen display, remote control, or any other device operative to interact with the computer system 500.

The computer system 500 also includes the drive unit 506 implemented as a disk or optical drive. The drive unit 506 includes a computer-readable medium 522 in which one or more sets of instructions 524, e.g. software, is embedded. Further, the sets of instructions 524 embodies one or more of the methods or logic as described herein. The sets of instructions 524 resides completely or partially within the memory 504 and/or within the processor 502 during execution by the computer system 500. The memory 504 and the processor 502 also include computer-readable media as discussed above.

In some systems, computer-readable medium 522 includes the set of instructions 524 or receives and executes the set of instructions 524 responsive to a propagated signal so that a device connected to network 530 communicates voice, video, audio, images, or any other data over the network 530. Further, the sets of instructions 524 are transmitted or received over the network 530 via the communication port or interface 520, and/or using the bus 508. The communication port or interface 520 is a part of the processor 502 or is a separate component. The communication port or interface 520 is created in software or is a physical connection in hardware. The communication port or interface 520 is configured to connect with the network 530, external media, the display 510, or any other components in the computer system 500, or combinations thereof. The connection with the network 530 is a physical connection, such as a wired Ethernet connection, or is established wirelessly as discussed below. Likewise, the additional connections with other components of the computer system 500 are physical connections or are established wirelessly. The network 530 alternatively be directly connected to the bus 508.

While the computer-readable medium 522 is shown to be a single medium, the term “computer-readable medium” includes a single medium or multiple media, such as a centralized or distributed database, and/or associated caches and servers that store one or more sets of instructions. The term “computer-readable medium” also includes any medium that is capable of storing, encoding, or carrying a set of instructions for execution by a processor or that causes a computer system to perform any one or more of the methods or operations disclosed herein. The computer-readable medium 922 is non-transitory and tangible.

The computer-readable medium 522 includes a solid-state memory such as a memory card or other package that houses one or more non-volatile read-only memories. The computer-readable medium 522 is a random-access memory or other volatile re-writable memory. Additionally or alternatively, the computer-readable medium 522 includes a magneto-optical or optical medium, such as a disk or tapes or other storage device to capture carrier wave signals such as a signal communicated over a transmission medium. A digital file attachment to an e-mail or other self-contained information archive or set of archives is considered a distribution medium that is a tangible storage medium. Accordingly, the disclosure is considered to include any one or more of a computer-readable medium or a distribution medium and other equivalents and successor media, in which data or instructions are stored.

In an alternative implementation, dedicated hardware implementations, such as application specific integrated circuits, programmable logic arrays, and other hardware devices, is constructed to implement one or more of the methods described herein. Applications that include the apparatus and systems of various implementations broadly include a variety of electronic and computer systems. One or more implementations described herein implement functions using two or more specific interconnected hardware modules or devices with related control and data signals that are communicated between and through the modules, or as portions of an application-specific integrated circuit. Accordingly, the present system encompasses software, firmware, and hardware implementations.

Computer system 500 is connected to the network, e.g., network 140. The network 140 defines at least one network including wired or wireless networks, e.g., network 140. The wireless network is a cellular telephone network, an 802.10, 802.16, 802.20, or WiMAX network. Further, such networks include a public network, such as the Internet, a private network, such as an intranet, or combinations thereof, and utilizes a variety of networking protocols now available or later developed including, but not limited to TCP/IP based networking protocols. The network 140 includes wide area networks (WAN), such as the Internet, local area networks (LAN), campus area networks, metropolitan area networks, a direct connection such as through a Universal Serial Bus (USB) port, or any other networks that allows for data communication. The network 140 is configured to couple one computing device to another computing device to enable communication of data between the devices. The network 140 is generally enabled to employ any form of machine-readable media for communicating information from one device to another. The network 140 includes communication methods by which information travels between computing devices. The network 140 is divided into sub-networks. The sub-networks allow access to all of the other components connected thereto or the sub-networks restrict access between the components. The network 140 is regarded as a public or private network connection and includes, for example, a virtual private network or an encryption or other security mechanism employed over the public Internet, or the like.

In accordance with various implementations of the present disclosure, the methods described herein are implemented by software programs executable by a computer system. Further, in an example, non-limited implementation, implementations can include distributed processing, component/object distributed processing, and parallel processing. Alternatively, virtual computer system processing can be constructed to implement one or more of the methods or functionality as described herein.

Although the present specification describes components and functions that are implemented in particular implementations with reference to particular standards and protocols, the disclosure is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosure is not limited to any particular implementation or programming technique and that the disclosure is implemented using any appropriate techniques for implementing the functionality described herein. The disclosure is not limited to any particular programming language or operating system.

It should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention are practiced without these specific details. In other instances, well-known methods, structures and techniques have not been shown in detail in order not to obscure an understanding of this description.

Thus, while there has been described what are believed to be the preferred embodiments of the invention, those skilled in the art will recognize that other and further modifications are made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

The present disclosure furthermore relates to the following aspects.

Example 1. A computer-implemented method comprising: obtaining, by one or more processors, hospital admission data associated with a plurality of individuals, the hospital admission data including at least one indicator of a disease of interest and at least one admission date; determining, by the one or more processors, a primary admission value based on the hospital admission data; determining, by the one or more processors, a readmission value based on the hospital admission data; determining, by the one or more processors, a disease-specific readmission rate based on the primary admission value and the readmission value, wherein the primary admission value and the readmission value are based on a common indicator of a disease of interest; and causing to output, by the one or more processors, data associated with the disease-specific readmission rate via a graphical user interface of a user device.

Example 2. The method of example 1, wherein determining the primary admission value based on the hospital admission data includes: determining at least one primary admission in at least one first time frame of interest, wherein each of the at least one first time frame of interest has zero or one primary admission; and determining a total number of primary admissions in a second time frame of interest, wherein the second time frame of interest includes the at least one first time frame of interest.

Example 3. The method of example 2, wherein determining the readmission value based on the hospital admission data includes: determining a total number of readmissions associated with the at least one primary admission in the second time frame of interest.

Example 4. The computer-implemented method of any of the preceding examples, wherein the hospital admission data further includes at least one of: an International Classification of Diseases and Related Health Problems (ICD) diagnosis code; a Clinical Care Document (CCD) summary; Admission, Discharge, Transfer (ADT) data; a Health Level Seven (HL7) message; an individual medical record number; an individual demographical information; insurance claims data; an admitting hospital location; an individual residence location; or prior discharge data.

Example 5. The computer-implemented method of any of the preceding examples, wherein determining the primary admission value comprises: determining, by the one or more processors, whether any of the plurality of individuals has died within a third time frame of interest; and upon determining at least one of the plurality of individuals has died, removing, by the one or more processors, data associated with the at least one of plurality of individuals that has died from the hospital admission data.

Example 6. The computer-implemented method of any of the preceding examples, wherein determining the disease-specific readmission rate comprises: dividing the readmission value by the primary admission value; or determining, using a trained first machine learning model, the disease-specific readmission rate based on at least a portion of the hospital admissions data associated with an individual, wherein the trained first machine learning model has been trained by: receiving, as disease-specific readmission rate training data, the hospital admission data including a plurality of admission dates associated with a plurality of users and a plurality of indicators of diseases of interest corresponding to the plurality of admission dates, and training a first machine learning model, using the disease-specific readmission rate training data, to infer the disease-specific readmission rate.

Example 7. The computer-implemented method of any of the preceding examples, wherein the primary admission value is determined based on one indicator of a disease of interest.

Example 8. The computer-implemented method of example 7, wherein the readmission value is determined based on the one indicator of a disease of interest.

Example 9. The computer-implemented method of any of the preceding examples, wherein the primary admission value and the readmission value are determined based on a first time frame of interest, wherein the first time frame of interest is a time frame beginning at a primary admission.

Example 10. The computer-implemented method of any of the preceding examples, wherein the primary admission value and the readmission value are determined based on a first time frame of interest and the disease-specific readmission rate is determined based on a second time frame of interest, the first time frame of interest being different from the second time frame of interest.

Example 11. The computer-implemented method of any of the preceding examples, further comprising: generating, by the one or more processors, at least one trend prediction for an individual based on the disease-specific readmission rate, the at least one trend prediction for an individual including at least one of a readmission prediction, a disease progression prediction, a disease prognosis prediction, or a cost prediction, wherein the data associated with the disease-specific readmission rate includes the at least one trend prediction.

Example 12. The computer-implemented method of example 11, wherein generating the at least one trend prediction for an individual includes: obtaining trend data, the trend data including at least one of disease of interest readmission data, other disease readmission data, readmission cost data, or prognosis data; and determining, using a trained second machine learning model, the at least one trend prediction based on the trend data, wherein the trained second machine learning model has been trained by: receiving, as trend prediction training data, at least one of disease of interest readmission data, other disease readmission data, readmission cost data, or prognosis data associated with a plurality of users; and training a machine learning model, using the trend prediction training data, to infer at least one trend in disease-specific readmission for an individual.

Example 13. The computer-implemented method of any of the preceding examples, further comprising: determining, by the one or more processors, a disease-any-cause readmission rate based on the primary admission value and the readmission value, wherein the primary admission value, the readmission value, and the disease-any-cause readmission rate are based on a plurality of indicators of diseases of interest.

Example 14. A system comprising: one or more storage devices each configured to store instructions; and one or more processors configured to execute the instructions to perform operations comprising: obtaining hospital admission data associated with a plurality of individuals, the hospital admission data including at least one indicator of a disease of interest and at least one admission date; determining a primary admission value based on the hospital admission data; determining a readmission value based on the hospital admission data; determining a disease-specific readmission rate based on the primary admission value and the readmission value, wherein the primary admission value and the readmission value are based on a common indicator of a disease of interest; and causing to output data associated with the disease-specific readmission rate via a graphical user interface of a user device.

Example 15. The system of example 14, wherein determining the primary admission value based on the hospital admission data includes: determining at least one primary admission in at least one first time frame of interest, wherein each of the at least one first time frame of interest has zero or one primary admission; and determining a total number of primary admissions in a second time frame of interest, wherein the second time frame of interest includes the at least one first time frame of interest.

Example 16. The system of example 15, wherein determining the readmission value based on the hospital admission data includes: determining a total number of readmissions associated with the at least one primary admission in the second time frame of interest.

Example 17. The system of example 14, 15, or 16, wherein determining the primary admission value comprises: determining, by the one or more processors, whether any of the plurality of individuals has died within a third time frame of interest; and upon determining at least one of the plurality of individuals has died, removing, by the one or more processors, data associated with the at least one of plurality of individuals that has died from the hospital admission data.

Example 18. The system of example 14, 15, 16, or 17, the operations further comprising: generating at least one trend prediction for an individual based on the disease-specific readmission rate, the at least one trend prediction for an individual including at least one of a readmission prediction, a disease progression prediction, a disease prognosis prediction, or a cost prediction, wherein the data associated with the disease-specific readmission rate includes the at least one trend prediction.

Example 19. The system of example 14, 15, 16, 17, or 18, the operations further comprising: dividing the readmission value by the primary admission value to determine the disease-specific readmission rate; and determining a disease-any-cause readmission rate based on the primary admission value and the readmission value, wherein the primary admission value, the readmission value, and the disease-any-cause readmission rate are based on a plurality of indicators of diseases of interest.

Example 20. A non-transitory computer-readable medium comprising instructions that, when executed by one or more processors, cause the one or more processors to perform operations, the operations comprising: obtaining hospital admission data associated with a plurality of individuals, the hospital admission data including at least one indicator of a disease of interest and at least one admission date; determining a primary admission value based on the hospital admission data; determining a readmission value based on the hospital admission data; determining a disease-specific readmission rate based on the primary admission value and the readmission value, wherein the primary admission value and the readmission value are based on a common indicator of a disease of interest; and causing to output data associated with the disease-specific readmission rate via a graphical user interface of a user device.

We claim:

1. A computer-implemented method comprising:
obtaining, by one or more processors, hospital admission data associated with a plurality of individuals, the hospital admission data including at least one indicator of a disease of interest and at least one admission date;
determining, by the one or more processors, a primary admission value based on the hospital admission data;
determining, by the one or more processors, a readmission value based on the hospital admission data;
determining, by the one or more processors, a disease-specific readmission rate based on the primary admission value and the readmission value,
wherein the primary admission value and the readmission value are based on a common indicator of a disease of interest; and
causing to output, by the one or more processors, data associated with the disease-specific readmission rate via a graphical user interface of a user device.

2. The method of claim 1, wherein determining the primary admission value based on the hospital admission data includes:
determining at least one primary admission in at least one first time frame of interest, wherein each of the at least one first time frame of interest has zero or one primary admission; and
determining a total number of primary admissions in a second time frame of interest, wherein the second time frame of interest includes the at least one first time frame of interest.

3. The method of claim 2, wherein determining the readmission value based on the hospital admission data includes:
determining a total number of readmissions associated with the at least one primary admission in the second time frame of interest.

4. The computer-implemented method of claim 1, wherein the hospital admission data further includes at least one of: an International Classification of Diseases and Related Health Problems (ICD) diagnosis code; a Clinical Care Document (CCD) summary; Admission, Discharge, Transfer (ADT) data; a Health Level Seven (HL7) message; an individual medical record number; an individual demographical information; insurance claims data; an admitting hospital location; an individual residence location; or prior discharge data.

5. The computer-implemented method of claim 1, wherein determining the primary admission value comprises:
determining, by the one or more processors, whether any of the plurality of individuals has died within a third time frame of interest; and
upon determining at least one of the plurality of individuals has died, removing, by the one or more processors, data associated with the at least one of plurality of individuals that has died from the hospital admission data.

6. The computer-implemented method of claim 1, wherein determining the disease-specific readmission rate comprises:
dividing the readmission value by the primary admission value; or
determining, using a trained first machine learning model, the disease-specific readmission rate based on at least a portion of the hospital admissions data associated with an individual, wherein the trained first machine learning model has been trained by:
receiving, as disease-specific readmission rate training data, the hospital admission data including a plurality of admission dates associated with a plurality of users and a plurality of indicators of diseases of interest corresponding to the plurality of admission dates, and
training a first machine learning model, using the disease-specific readmission rate training data, to infer the disease-specific readmission rate.

7. The computer-implemented method of claim 1, wherein the primary admission value is determined based on one indicator of a disease of interest.

8. The computer-implemented method of claim 7, wherein the readmission value is determined based on the one indicator of a disease of interest.

9. The computer-implemented method of claim 1, wherein the primary admission value and the readmission value are determined based on a first time frame of interest, wherein the first time frame of interest is a time frame beginning at a primary admission.

10. The computer-implemented method of claim 1, wherein the primary admission value and the readmission value are determined based on a first time frame of interest and the disease-specific readmission rate is determined based on a second time frame of interest, the first time frame of interest being different from the second time frame of interest.

11. The computer-implemented method of claim 1, further comprising:
generating, by the one or more processors, at least one trend prediction for an individual based on the disease-specific readmission rate, the at least one trend prediction for an individual including at least one of a readmission prediction, a disease progression prediction, a disease prognosis prediction, or a cost prediction,
wherein the data associated with the disease-specific readmission rate includes the at least one trend prediction.

12. The computer-implemented method of claim 11, wherein generating the at least one trend prediction for an individual includes:
obtaining trend data, the trend data including at least one of disease of interest readmission data, other disease readmission data, readmission cost data, or prognosis data; and
determining, using a trained second machine learning model, the at least one trend prediction based on the trend data, wherein the trained second machine learning model has been trained by:
receiving, as trend prediction training data, at least one of disease of interest readmission data, other disease readmission data, readmission cost data, or prognosis data associated with a plurality of users; and
training a machine learning model, using the trend prediction training data, to infer at least one trend in disease-specific readmission for an individual.

13. The computer-implemented method of claim 1, further comprising:
determining, by the one or more processors, a disease-any-cause readmission rate based on the primary admission value and the readmission value,
wherein the primary admission value, the readmission value, and the disease-any-cause readmission rate are based on a plurality of indicators of diseases of interest.

14. A system comprising:
one or more storage devices each configured to store instructions; and
one or more processors configured to execute the instructions to perform operations comprising:
obtaining hospital admission data associated with a plurality of individuals, the hospital admission data including at least one indicator of a disease of interest and at least one admission date;
determining a primary admission value based on the hospital admission data;
determining a readmission value based on the hospital admission data;
determining a disease-specific readmission rate based on the primary admission value and the readmission value,
wherein the primary admission value and the readmission value are based on a common indicator of a disease of interest; and
causing to output data associated with the disease-specific readmission rate via a graphical user interface of a user device.

15. The system of claim 14, wherein determining the primary admission value based on the hospital admission data includes:
determining at least one primary admission in at least one first time frame of interest, wherein each of the at least one first time frame of interest has zero or one primary admission; and
determining a total number of primary admissions in a second time frame of interest, wherein the second time frame of interest includes the at least one first time frame of interest.

16. The system of claim 15, wherein determining the readmission value based on the hospital admission data includes:
determining a total number of readmissions associated with the at least one primary admission in the second time frame of interest.

17. The system of claim 14, wherein determining the primary admission value comprises:
determining, by the one or more processors, whether any of the plurality of individuals has died within a third time frame of interest; and
upon determining at least one of the plurality of individuals has died, removing, by the one or more processors, data associated with the at least one of plurality of individuals that has died from the hospital admission data.

18. The system of claim 14, the operations further comprising:
generating at least one trend prediction for an individual based on the disease-specific readmission rate, the at least one trend prediction for an individual including at least one of a readmission prediction, a disease progression prediction, a disease prognosis prediction, or a cost prediction,
wherein the data associated with the disease-specific readmission rate includes the at least one trend prediction.

19. The system of claim 14, the operations further comprising:
dividing the readmission value by the primary admission value to determine the disease-specific readmission rate; and
determining a disease-any-cause readmission rate based on the primary admission value and the readmission value,
wherein the primary admission value, the readmission value, and the disease-any-cause readmission rate are based on a plurality of indicators of diseases of interest.

20. A non-transitory computer-readable medium comprising instructions that, when executed by one or more processors, cause the one or more processors to perform operations, the operations comprising:
obtaining hospital admission data associated with a plurality of individuals, the hospital admission data including at least one indicator of a disease of interest and at least one admission date;
determining a primary admission value based on the hospital admission data;
determining a readmission value based on the hospital admission data;
determining a disease-specific readmission rate based on the primary admission value and the readmission value,
wherein the primary admission value and the readmission value are based on a common indicator of a disease of interest; and
causing to output data associated with the disease-specific readmission rate via a graphical user interface of a user device.
